(19) United States
(12) Patent Application Publication
(10) Pub. No.: US 2011/0071096 A1
(43) Pub. Date: Mar. 24, 2011

(54) Macrolides Having Antibiotic Activity

(75) Inventors: Murthy V.S.N. Yerramilli, Falmouth, ME (US); Michael Randolph Atkinson, Cape Elizabeth, ME (US); Hengguang Ll, Morrisville, NC (US); Christopher Alan Cox, Raleigh, NC (US)

(73) Assignee: IDEXX Laboratories, Inc., Westbrook, ME (US)

(21) Appl. No.: 12/859,597

(22) Filed: Aug. 19, 2010

Related U.S. Application Data
(60) Provisional application No. 61/244,908, filed on Sep. 23, 2009.

(51) Int. Cl. A61K 31/7048; C07H 17/08; A61P 31/00; A61P 31/04

(52) U.S. Cl. 514/28; 536/7.1

(57) ABSTRACT

The present invention relates to a semi-synthetic macrolide having antimicrobial activity, in particular antibacterial activity; pharmaceutical compositions comprising the macrolide; and methods of using the macrolide to treat or prevent an infection.

### FIELD OF THE INVENTION

The present invention relates to semi-synthetic macrolides having antimicrobial activity, in particular antibacterial activity; pharmaceutical compositions comprising the macrolide; and methods of using the macrolide to treat or prevent an infection.

### BACKGROUND OF THE INVENTION

Macrolides are class of antibacterial agents that are useful against a wide spectrum of bacterial organisms and are routinely used to treat infections such as respiratory tract infections and soft tissue infections. In addition to antibacterial activity, macrolides may also possess antimicrobial, antitumor, and anti-inflammatory properties. See, e.g., U.S. Pat. No. 5,795,871 and WO 2006/087644.

Macrolide antibiotics usually include one or more deoxysugars (e.g., cladinose and desosamine) attached to a lactone ring (e.g., a 14, 15, or 16-membered ring). Exemplary macrolides include erythromycin, clarithromycin, roxithromycin, dirithromycin, azithromycin, desmycosin, and lactenocin. Macrolides are believed to inhibit bacterial protein synthesis by binding reversibly to subunit 505 of the bacterial ribosome, thereby inhibiting translocation of peptidyl-tRNA. This action is mainly bacteriostatic, but can also be bactericidal.

Clinical use of macrolide antibiotics, however, has resulted in the evolution of bacterial strains that are resistant to the antibiotics. In an effort to target these resistant strains, new macrolide antibiotics have been developed by chemically modifying the structure of known macrolide antibiotics. The evolution of bacterial strains that are resistant to known macrolide antibiotics has resulted in a continual need for new macrolide antibiotics.

Furthermore, some macrolide antibiotics, after being administered to an subject, do not persist in the subject's blood stream or tissue at a therapeutically effective level for a long enough period of time to have a therapeutic effect. In these cases, frequent dosing is necessary to maintain a therapeutically effective blood and/or tissue level of the antibiotic. Such frequent dosing, however, is labor intensive and costly, especially in the case of meat-producing animals. In other cases, the antibiotic is poorly tolerated by, or toxic to, the subject at a therapeutically effective dose. Accordingly, there is a continual need for new macrolide antibiotics, in particular, macrolide antibiotics with increased potency, longer half-life, and/or increased therapeutic index.

### SUMMARY OF THE INVENTION

The invention is directed to semi-synthetic macrolides. In one embodiment, the macrolide is a compound of Formula 1;

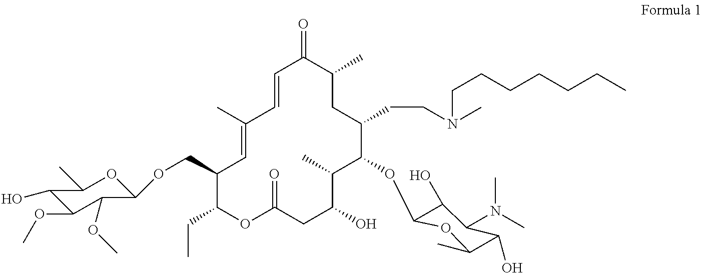

In one embodiment, the semi-synthetic macrolide is a compound of formula 2:

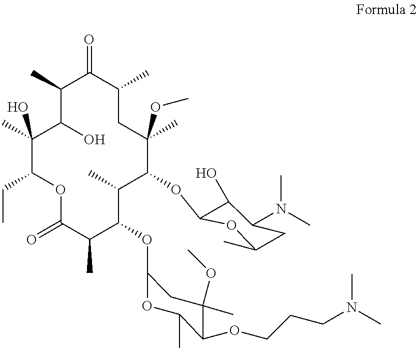

In one embodiment, the semi-synthetic macrolide is a compound of formula 3:

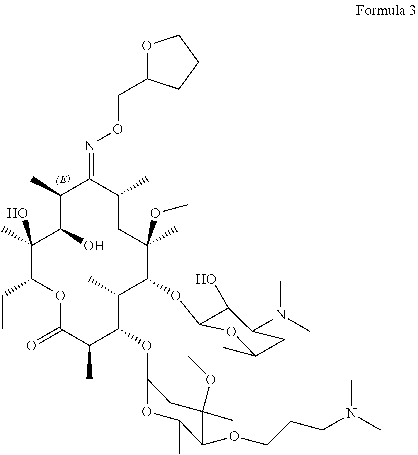

In one embodiment, the semi-synthetic macrolide is a compound of formula 4:

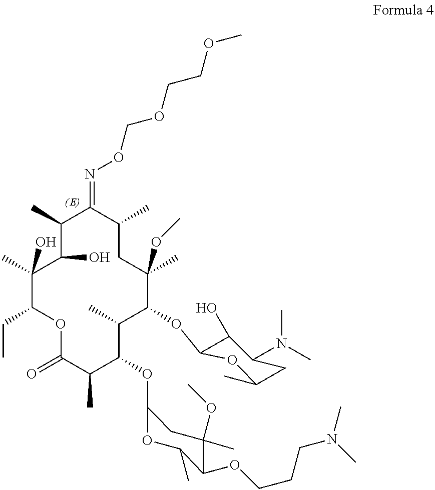

In one embodiment, the semi-synthetic macrolide is a compound of formula 5:

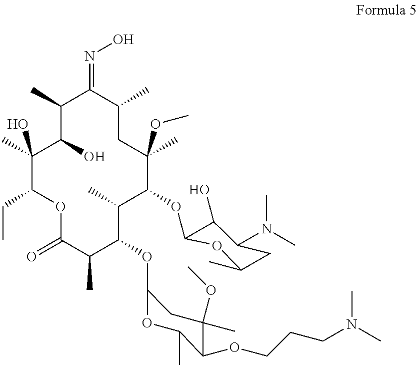

In one embodiment, the semi-synthetic macrolide is a compound of formula 6:

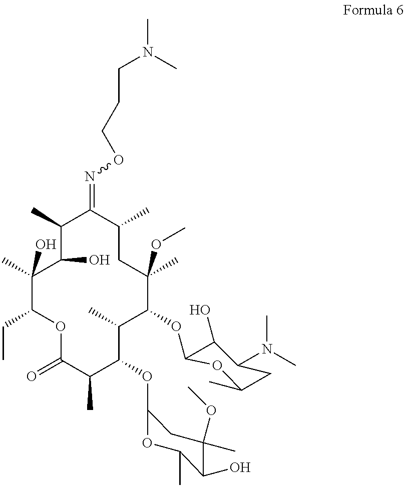

The invention also encompasses pharmaceutically acceptable salts of the compounds of formula 1-6.

The invention is further directed to a method of treating or preventing an infection, e.g., a bacterial infection, in an animal comprising administering a compound of formula 1-6 to the animal.

The invention is further directed to a pharmaceutical composition comprising a compound of formula 1-6 and at least one pharmaceutically acceptable excipient.

### BRIEF DESCRIPTION OF THE DRAWINGS

FIG. 1 depicts the serum concentration of the compound of formula 2 as a function of time when the compound of formula 2 is administered to a dog.

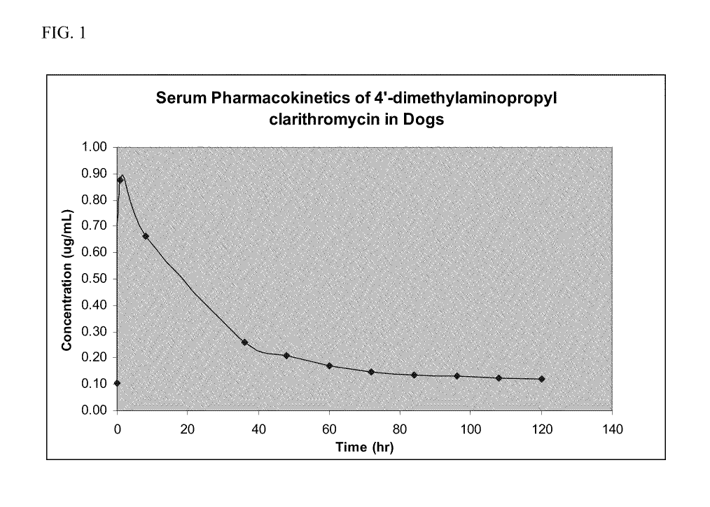

FIG. 2 depicts the concentration of the compound of formula 2 in various organs after the compound of formula 2 is administered to a dog.

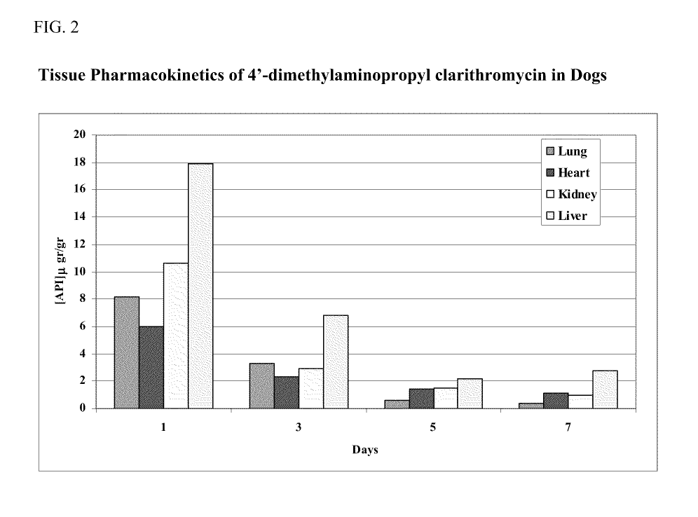

FIG. 3 depicts the serum concentration of the compound of formula 1 as a function of time when the compound of formula 1 is administered to a cat.

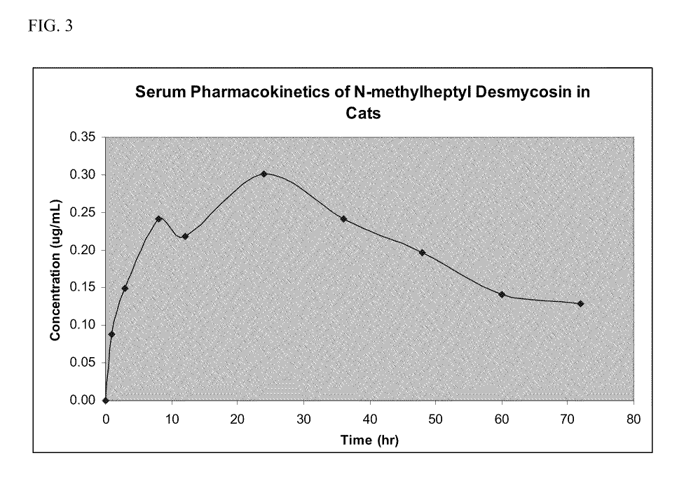

FIG. 4 depicts the concentration of the compound of formula 1 in various organs after the compound of formula 1 is administered to a cat.

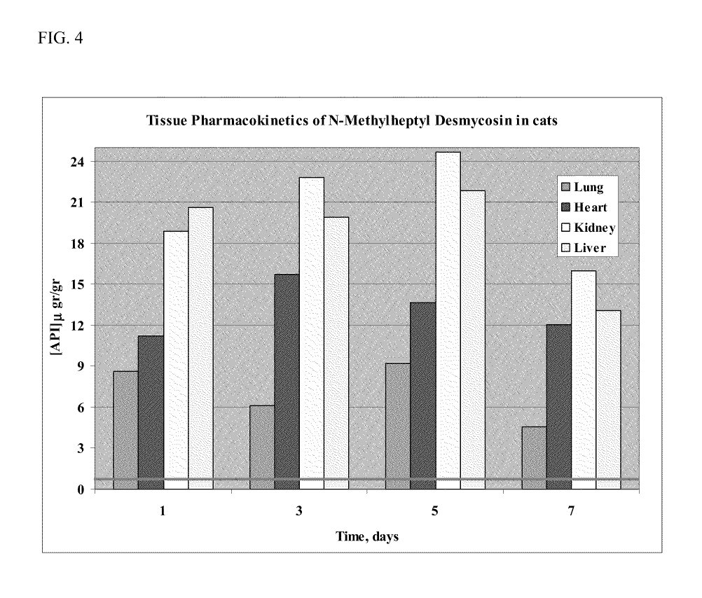

### DETAILED DESCRIPTION OF THE INVENTION

The invention is directed to semi-synthetic macrolides of formula 1-6, pharmaceutical compositions comprising a compound of formula 1-6, and methods of treating or preventing an infection, e.g., a bacterial infection, in an animal comprising administering a compound of formula 1-6 to the animal.

### DEFINITIONS

As used herein, the following terms have the following meaning:

The term “animal” includes, but is not limited to, humans, canines, felines, equines, bovines, ovines, porcines, amphibians, reptiles, and avians. Representative animals include, but are not limited to a cow, a horse, a sheep, a pig, an ungulate, a chimpanzee, a monkey, a baboon, a chicken, a turkey, a mouse, a rabbit, a rat, a guinea pig, a dog, a cat, and a human.

The phrase “effective amount” when used in connection with a compound of formula 1-6 means an amount for treating or preventing a bacterial infection.

The phrase “treating,” “treatment of,” and the like includes the amelioration or cessation of a specified condition, typically a bacterial infection.

The phrase “preventing,” “prevention of,” and the like include the avoidance of the onset of a condition, typically a bacterial infection.

The phrase “pharmaceutically acceptable salt,” as used herein, is a salt formed from a basic nitrogen group of a compound of formula 1-6 and an acid. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, pamoate (i.e., 1,1′-methylene-bis-(2-hydroxy-3-naphthoate)), decanoate, laurate, myristate, and palmitate salts.

The phrase “single dose,” as used herein means a dose that is administered only once over a 28-day period. The dose may be administered in a single dosage form, such as a single injection or one capsule or tablet, or may be divided, e.g. constituted by more than one dosage form, such as by multiple capsules or tablets that are taken at or about the same time. The single dose is effective at treating or preventing a bacterial infection in an animal in need thereof. The “single dose” used in the methods of the invention is formulated for immediate release and is not formulated for controlled or sustained release. For example, an orally administered single dose of a compound of formula 1-6 is preferably administered in a form such that it releases the compound of formula 1-6 to the gastrointestinal tract of the animal at a rate such that the total amount of the compound of formula 1-6 is released from the dosage form in less than about 60 minutes.

In the interest of brevity, unless otherwise specified, the phrase “compound of formula 1,” and similar phrases for compounds of formula 2-6 encompass, both the compound of formula 1 (formula 2-6) and pharmaceutically acceptable salts of the compound of formula 1 (formula 2-6).

Any numerical values recited herein include all values from the lower value to the upper value in increments of any measurable degree of precision. For example, if the value of a variable such as weight percent, dosing amounts, dosing regimens, and the like is 1 to 90, specifically from 20 to 80, and more specifically from 30 to 70, it is intended that values such as 15 to 85, 22 to 68, 43 to 51, 30.3 to 32, etc., are expressly enumerated in this specification. In other words, all possible combinations of numerical values between the lowest value and the highest value enumerated are to be considered to be expressly stated in this application in a similar manner.

### Synthesis of the Compounds of Formula 1-6

The compound of formula 1 can be prepared by the following illustrative route of synthesis:

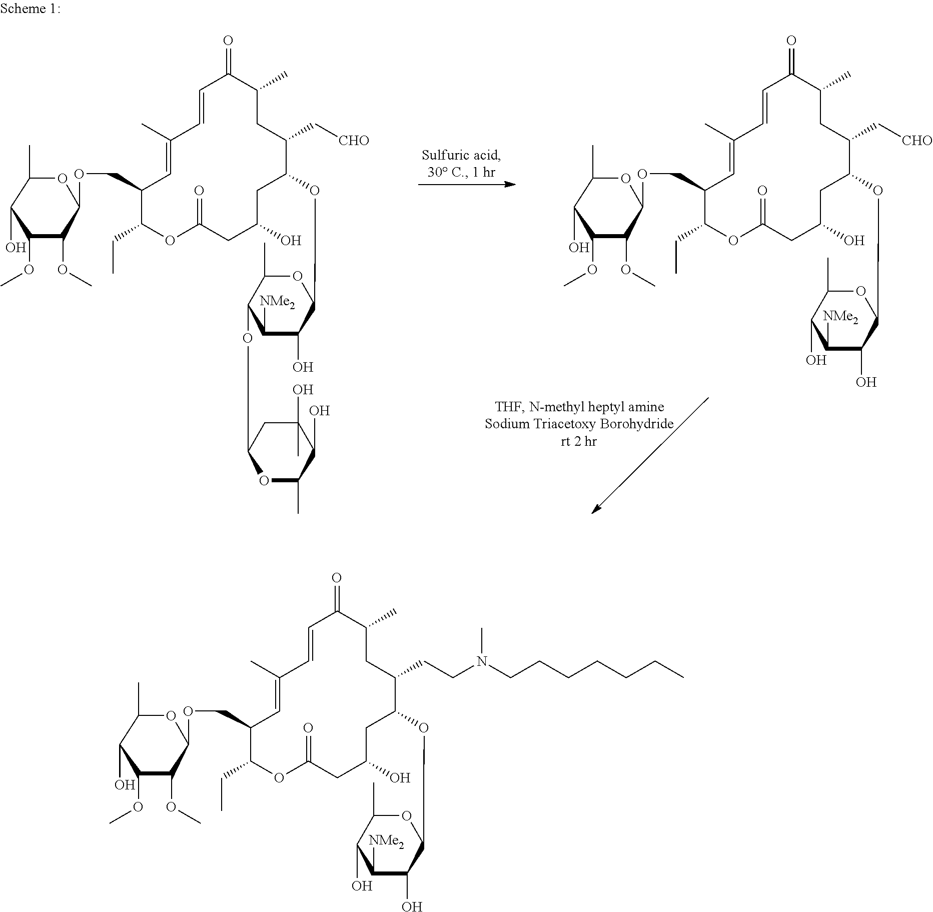

Example 1 provides a representative synthesis of the compound of formula 1.

The compound of formula 2 can be prepared by the following illustrative route of synthesis:

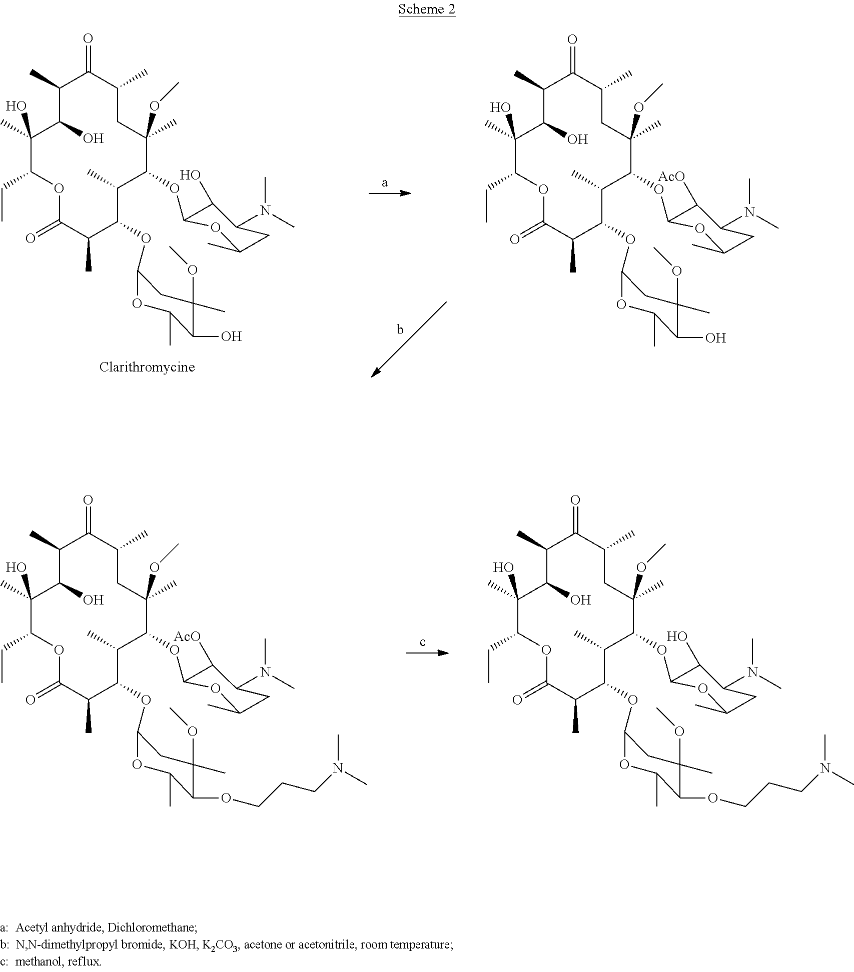

Example 2 provides a representative synthesis of the compound of formula 2.

The compound of formula 3 can be prepared by the following illustrative route of synthesis:

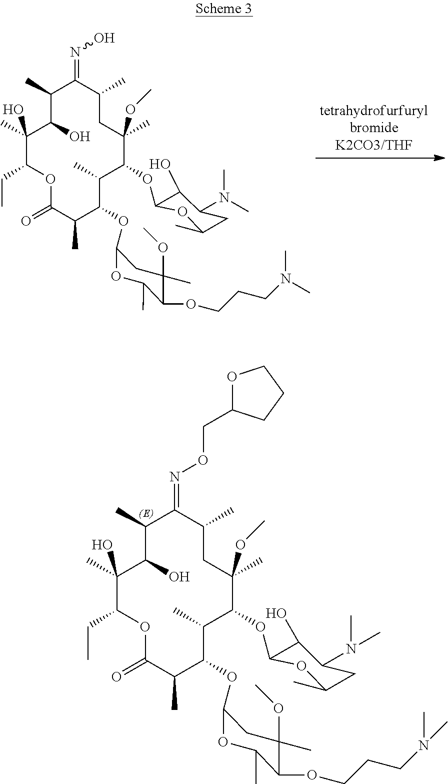

Example 3 provides a representative synthesis of the compound of formula 3.

The compounds of formula 4 and 5 can be prepared by the following illustrative route of synthesis:

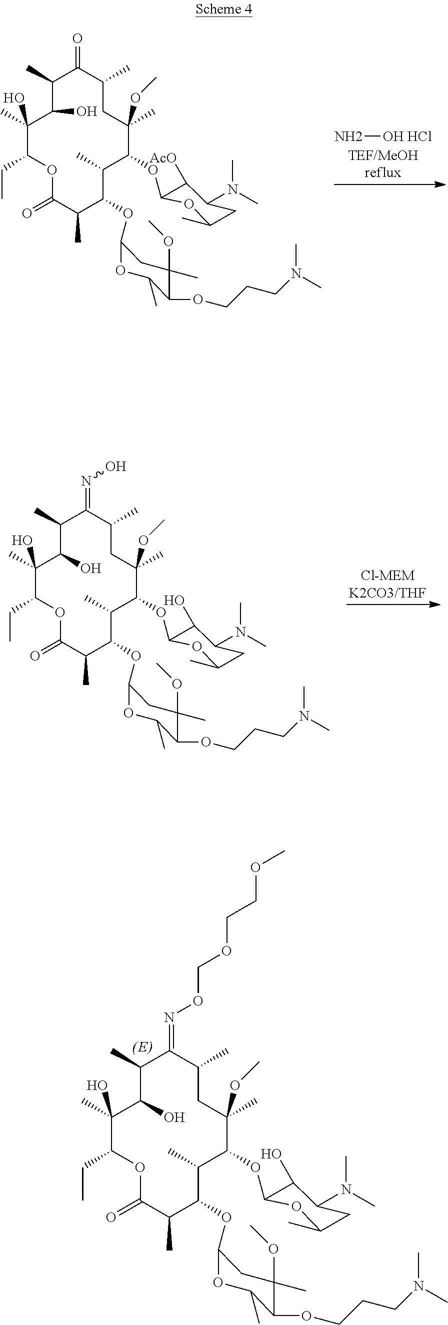

Example 4 provides a representative synthesis of the compounds of formula 4 and 5.

The compound of formula 6 can be prepared by the following illustrative route of synthesis:

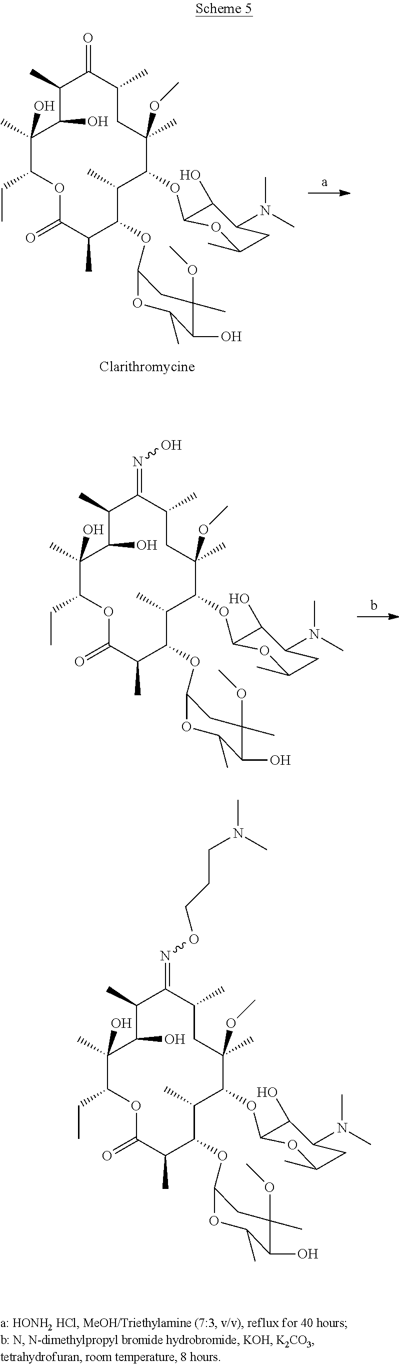

Example 5 provides a representative synthesis of the compound of formula 6.

The macrolide antibiotic can be purified using standard methods known in the art including, but not limited to, recrystallization, extraction, and chromatography.

The salts of the compounds of formula 1-6 can be prepared by simply contacting a compound of formula 1-6 and an acid. Typically, the compound of formula 1-6 is contacted with the acid by dissolving the compounds of formula 1-6 in a suitable solvent to provide a solution and adding at least one equivalent of acid per equivalent of the compound of formula 1-6 to the resulting solution. Typically, about 0.9 to 3, preferably about 0.95 to 2.5, more preferably about 1 to 2.3, and most preferably about 1 to 2.1 equivalents of acid is used per equivalent of the compound of formula 1-6. In one embodiment, about 0.9 to 1.2 equivalents of acid is used per equivalent of the compound of formula 1-6. Typically, the acid is added to the solution with stirring. Typically, the solvent is at room temperature, however, the solvent can be heated to any temperature up to the boiling point of the solvent, provided that the elevated temperature does not cause decomposition of the compound of formula 1-6 or the acid. Preferably, the solvent dissolves both the compounds of formula 1-6 and the acid. In one embodiment, the solvent is a non-aqueous solvent. In one embodiment, the solvent is an organic solvent. In one embodiment, the solvent is water. Illustrative solvents useful for preparing the acid salt of the compound of formula 1-6 include, but are not limited to, dichloromethane, methylene chloride, methanol, ethanol, acetone, ethyl acetate, and acetonitrile. After the acid is added to the solution, the resulting salt formed between the compound of formula 1-6 and the acid is separated from the solution. In one embodiment, the salt formed between the compound of formula 1-6 and the acid precipitates and is collected by filtration. In another embodiment, the solvent is removed by evaporation, typically under reduced pressure, to provide the salt formed between the compound of formula 1-6 and the acid as a solid. In one embodiment, the salt is prepared as a liquid solution of the salt by contacting the compounds of formula 1-6 and the acid in a pharmaceutically acceptable solvent known to those skilled in the art, such as, but not limited to propylene glycol, glycerol formal, dimethylsulfoxide, or polyethylene glycol.

The acid salt of the compounds of formula 1-6 can be purified using standard methods known to those skilled in the art including, but not limited to, recrystallization, extraction, and chromatography.

### Methods of Treating or Preventing Infections

Due to its antimicrobial activity, the compounds of formula 1-6 are advantageously useful in veterinary and human medicine. Accordingly, a method of treating or preventing an infection comprises administering a compound of formula 1-6 to an animal in need thereof.

The compounds of formula 1-6 can be used to treat or prevent an infection in any animal, including, but is not limited to, humans, canines, felines, equines, bovines, ovines, porcines, amphibians, reptiles, and avians. In one embodiment, the animal is a mammal. In another embodiment, the animal is a human. In one particular embodiment, the animal is a meat-producing animal, e.g., a cow, pig, chicken, turkey, or fish. In another particular embodiment, the animal is a domestic or laboratory animal, e.g., ungulates such as a horse, sheep, goat, or pig; primates such as a chimpanzee, monkey, or baboon; rodents such as a mouse, rat, or guinea pig; as well as other animals such as rabbit, dog, or cat.

In one embodiment, the infection is a bacterial infection. The bacterial infection can be gram negative or gram positive. The compounds of formula 1-6 are especially effective against gram negative bacteria. In another embodiment, the compounds of formula 1-6 are used to treat or prevent an infection by a bacterial strain that is resistant to one or more antibiotics.

Exemplary genera and exemplary species susceptible to Compound 1 include, but are not limited to:

| Exemplary susceptible genera | Exemplary susceptible species |
|---|---|
| Actinobacillus | Actinobacillus pleuropneumoniae, Actinomyces pyogenes |
| Aeromonas | Aeromonas salmonicidia |
| | Bacillus subtilis |
| Bacteroides | Bacterioides melaninogenicus, Bacteroides fragilis |
| Bordetella | Bordetella bronchoseptica |
| Chlamydia | |
| Edwardsiella | Edwardsiella ictaluri |
| Enterobacter | Enterobacter cloacae |
| Enterococcus | Enterococcus faecalis, Enterococcus faecium |
| Escherichia | Escherichia faecalis, Escherichia coli |
| Fusobacterium | Fusobacterium necrophorum |
| Haemophilus | Haemophilus somnus, Haemophilus influenzae |
| Klebsiella | Klebsiella pneumonia, Klebsiella oxytoca, Klebsiella salmonella |
| Mannheimia | Mannhemia haemolytica |
| Moraxella | |
| Mycoplasma | Mycoplasma bovis, Mycoplasma dispar, Mycoplasma |
| | hyopneumoniae, Mycoplasma hyorhinis, Mycoplasma |
| | gallisepticum, Mycoplasma mycoides, Mycoplasma |
| | ovipneumonia |
| Pasteurella | Pasteurella haemolytica, Pasteurella multocida, Pasteurella |
| | haemolytica |
| | Peptococcus indolicus |
| Proteus | Proteus enterobacter, Proteus mirabillis |
| | Pseudomonas aeruginosa |
| Salmonella | Salmonella cholerasuis |
| Serratia | |
| Shigella | |
| Staphylococcus | Staphylococcus aureaus, Staphylococcus intermedius |
| Streptococcus | Streptococcus pyogenes, Streptococcus suis |
| Ureaplasma | |

In one embodiment, the bacterial infection is caused by Pasteurella haemolytica, Pasteurella multocida, Pasteurella haemolytica, Haemophilus somnus, Actinobacillus pleuropneumoniae, Actinomyces pyogenes, Pseudomonas aeruginosa, Klebsiella pneumonia, Klebsiella oxytoca, Escherichia faecalis, Escherichia coli, Staphylococcus aureaus, Staphylococcus intermedius, Enterococcus faecalis, Enterococcus faecium, Streptococcus pyogenes, Bacillus subtilis, Peptococcus indolicus, Mycoplasma bovis, Mycoplasma dispar, Mycoplasma hyopneumoniae, Mycoplasma hyorhinis, Mycoplasma gallisepticum, Mycoplasma mycoides, Mycoplasma ovipneumonia, Haemophilus influenzae, Klebsiella salmonella, Shigella, Proteus enterobacter, Enterobacter cloacae, Mannhemia haemolytica, Haemophilus somnus, Fusobacterium necrophorum, Bacterioides melaminogenicus, Proteus mirabillis, Streptococcus suis, Salmonella cholerasuis, Edwardsiella ictaluri, Aeromonas salmonicidia, Actinobaccilus pleuropneumoniae, and Bordetella bronchoseptica.

In one embodiment, the bacterial infection is caused by Escheria coli, Pasteurella multocida, Klebsiella Pneumonia, Bordetella bronchispetica, or Bacteroides fragilis.

Typically, the minimum inhibitory concentration of the compound of formula 1-6 against a specific bacteria is less than 10 μg/mL, preferably less than 5 μg/mL, more preferably less than 2 μg/mL, even more preferably less than 1 μg/mL, and most preferably less than 0.5 μg/mL.

The activity of the compounds of formula 1-6 against a bacteria is determined using standard dilution tests. For example, the minimum inhibitory concentrations can be determined using the disk diffusion susceptibility testing method described in Clinical Microbiology Procedures Handbook, volume 1, edited by Henry D. Isenberg, American Society for Microbiology, 1992, section 5.1 or the well known method of Bauer et al. “Antibiotic Susceptibility Testing by a Standardized Single Disc Method,” Amer. J. Clin. Pathol., 45, p. 493-496.

Table 1 provides minimum inhibitory concentrations for the compound of formula 2 against various bacteria.

TABLE 1
Gram Positive Bacteria

| | Minimum Inhibitory Concentration (μg/mL) | | | | | |
|---|---|---|---|---|---|---|
| | Staphylococcus | Bacillus | Streptococcus | Streptococcus | Clostridium | Enterococcus |
| Compound | aureus | subtilis | pyogenes | pneumonia | perfringens | faecalis |
| Clarithromycin | <0.25 | <0.25 | 0.06⁽¹⁾ | 0.03⁽¹⁾ | 4* | 2.7⁽³⁾ |
| Erythromycin | 0.25 | 0.25 | 0.06⁽¹⁾ | 0.06⁽¹⁾ | 4* | 2.7⁽³⁾ |
| Roxithromycin | 0.25 | 0.25 | 0.25 | 0.25 | 2 | 8 |
| Formula 2 | 0.25 | 0.25 | 0.25 | 0.25 | 2 | 8 |

Gram Negative Bacteria

| | Minimum Inhibitory Concentration (μg/mL) | | | | |
|---|---|---|---|---|---|
| | | Pasteurella | Klebsiella | Bordetella | Bacteroides |
| Compound | Escheria Coli | multocida | Pneumonia | bronchispetica | fragilis |
| Clarithromycin | 32 | 2* | | 4 | 16* |
| Erythromycin | 32 | 4* | 128⁽²⁾ | 4 | >8* |
| Roxithromycin | 32 | 1 | 32 | 1 | <2 |
| Formula 2 | 32 | 1 | 32 | 4 | 2 |

*J. F. Presscott, Antimicrobial Therapy in Veterinary Medicine, 3^{rd} ed.
⁽¹⁾J. Med. Chem., 44, 4137-4156, (2001)
⁽²⁾J. Antimicrob. Agents in Chemother., 35: 6, 1116-1126, (1991)
⁽³⁾J. Clin. Micro., 26: 11, 2415-2420 (1988)

The data in Table 1 show that the compound of formula 2 is as effective or more effective against the gram negative bacteria Escheria coli, Pasteurella multocida, Klebsiella Pneumonia, Bordetella bronchispetica, and Bacteroides fragilis than clarithromycin, erythromycin, or roxythromycin. The data in Table 1 also show that the compound of formula 2 is more effective against the gram positive bacteria Clostridium perfringens than clarithromycin, erythromycin, or roxythromycin and of similar effectiveness against Staphylococcus aureus and Bacillus subtilis. The results reported in Table 1 show that the compound of formula 2 is an effective antibiotic and, therefore, useful for treating or preventing bacterial infections in animals.

Without wishing to be bound by theory, it is believed that the efficacy of the compound of formula 2 against bacteria, in particular, gram negative bacteria, is the result of obtaining the proper balance between the hydrophilicity and hydrophobicity of the antibiotic.

When administered to an animal, the compounds of formula 1-6 are typically administered as a component of a composition that comprises a pharmaceutically acceptable carrier or excipient. The compounds of formula 1-6 can be administered by any convenient route, for example, orally, by infusion or bolus injection, by absorption through epithelial or mucocutaneous linings (e.g., oral, rectal, and intestinal mucosa, etc.) and can be administered together with another therapeutically active agent. Administration can be systemic or local. Various delivery systems are known, e.g., encapsulation in liposomes, microparticles, microcapsules, capsules, tablets, etc., and can be used to administer the compounds of formula 1-6.

The compounds of formula 1-6 can be administered by any route including both enteral and parenteral routes. Exemplary routes of administration include, but are not limited to, oral, buccal, sublingual, rectal, vaginal, infusion or bolus injection, intravenous, intramuscular, subcutaneous, intraperitoneal, intracerebral, epidural, inhalation, intranasal, intradermal, transdermal, transmucosal, or topical (particularly to the ears, nose, eyes, or skin). Administration can be local or systemic. Usually, administration releases the compound of formula 1-6 into the bloodstream. The appropriate route of administration can be determined by the practitioner. In one embodiment, the compound of formula 1-6 is administered orally. In another embodiment, the compound of formula 1-6 is administered by injection.

In one embodiment, an effective amount of a compound of formula 1-6 is administered. In one embodiment, the effective amount of the compound of formula 1-6 is divided into about 2 to 4 individual daily doses. In one embodiment, the effective amount of the compound of formula 1-6 is administered as a single dose.

In one embodiment, a compound of formula 1-6 is administered by simply mixing a compound of formula 1-6 with the animal's food.

In one embodiment, an effective amount of a compound of formula 1-6 is administered by injection.

In one embodiment, an effective amount of a compound of formula 1-6 is administered by subcutaneous injection.

In one embodiment, an effective amount of a compound of formula 1-6 is administered by intramuscular injection.

In one embodiment, an effective amount of a compound of formula 1-6 is administered intravenously.

In one embodiment, an effective amount of a compound of formula 1-6 is administered topically. Typically, topical compositions are applied from about 1 to 5 times each day until the bacterial infection is abated. In one embodiment, the topical compositions are applied about once each day. In one embodiment, the topical compositions are applied about twice each day. In one embodiment, the topical compositions are applied about three times each day. In one embodiment, the topical compositions are applied about four times each day. In one embodiment, the topical applications are applied for about 4 weeks. In one embodiment, the topical applications are applied for about 3 weeks. In one embodiment, the topical applications are applied for about 2 weeks. In one embodiment, the topical applications are applied for about 1 week.

In yet another embodiment, the compound of formula 1-6 is delivered in a controlled-release system or sustained-release system (see, e.g., Goodson, in Medical Applications of Controlled Release, supra, vol. 2, pp. 115-138 (1984)). Controlled- or sustained-release systems such as those discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)). In yet another embodiment, a controlled- or sustained-release system can be placed in proximity of a target of the compound of formula 1-6, thus requiring only a fraction of the systemic dose.

In specific embodiments, it can be desirable to administer the compound of formula 1-6 locally. This can be achieved, for example, and not by way of limitation, by local infusion during surgery, topical application, e.g., in conjunction with a wound dressing after surgery, by injection, or by means of an implant, said implant being of a porous, non-porous, or gelatinous material, including membranes, such as sialastic membranes or fibers.

The amount of the compound of formula 1-6 that is effective in the treatment or prevention of a bacterial infection can be determined by standard clinical techniques. In addition, in vitro or in vivo assays can optionally be employed to help identify optimal dosage ranges. The precise dose to be employed will also depend on the route of administration, the seriousness or severity of the bacterial infection, the susceptibility of the infecting organism to the compound of formula 1-6, and the characteristics of the animal being treated and can be decided according to the judgment of a practitioner and/or each animal's circumstances. Suitable effective dosage amounts, however, typically range from about 1 to 50 mg/kg/day. In one embodiment, the effective dosage amount ranges from about 5 to 30 mg/kg/day. In one embodiment, the effective dosage amount ranges from about 5 to 20 mg/kg/day.

In one embodiment, an effective dosage amount of a compound of formula 1-6 is administered about every 7 days until the bacterial infection is abated.

In one embodiment, an effective dosage amount of a compound of formula 1-6 is administered about every 7 days for 4 weeks.

In one embodiment, an effective dosage amount of a compound of formula 1-6 is administered about every 7 days for 2 weeks.

In one embodiment, an effective dosage amount of a compound of formula 1-6 is administered about every other day for 4 weeks.

In one embodiment, an effective dosage amount of a compound of formula 1-6 is administered about every other day for 2 weeks.

In one embodiment, an effective dosage amount of a compound of formula 1-6 is administered about every 3 days for 9 days.

In one embodiment, a single effective dosage amount of a compound of formula 1-6 is administered, i.e., a single dose of a compound of formula 1-6 is used to treat or prevent a bacterial infection.

It is believed that the compounds of formula 1-6 have a significantly slower rates of clearance than other macrolides, in particular clarithromycin or roxithromycin, when administered to an animal. The slower rate of clearance means that the compounds of formula 1-6 need to be administered less regularly. Indeed, a single dose of a compound of formula 1-6 is often effective at treating or preventing a bacterial infection in an animal. The ability to treat or prevent a bacterial infection with a single dose of a compound of formula 1-6 is an advantage in that a single dose is more convenient, less costly, and provides better patient compliance.

In one embodiment, the method involves treating or preventing a bacterial infection in an animal comprising administering to an animal in need thereof a single dose of an effective amount of a compound of formula 1-6.

In one embodiment, a single effective dosage amount of a compound of formula 1-6 is administered orally.

In one embodiment, a single effective dosage amount of a compound of formula 1-6 is administered by injection.

In one embodiment, a single effective dosage amount of a compound of formula 1-6 is administered by subcutaneous injection.

In one embodiment, a single effective dosage amount of a compound of formula 1-6 is administered by intramuscular injection.

In one embodiment, a single effective dosage amount of a compound of formula 1-6 is administered intravenously.

In one embodiment, a single effective dosage amount of a compound of formula 1-6 is administered topically.

In one embodiment, the animal is a mammal.

In one embodiment, the animal is a human.

In one embodiment, the animal is a dog.

In one embodiment, the animal is a cat.

In one embodiment, the animal is a cow.

In one embodiment, the animal is a pig.

In one embodiment, the animal is a horse.

The present methods for treating or preventing a bacterial infection in an animal in need thereof can further comprise administering another therapeutic agent to the animal being administered a compound of formula 1-6. In one embodiment, the other therapeutic agent is administered in an effective amount.

Other therapeutic agents includes, but are not limited to, other antibiotics, antifungal agents, antiviral agents, antiparasitic agents, and anti-inflammatory agents.

Examples of useful antibiotics include, but are not limited to, amoxicillin; ampicillin; ceftiofor; erythromycin; oxytetracycline; procaine penicillin G; sulfonamides; tylosin; tilmicosin; cephalosporins; chloramphenicol; aminoglycosides, such as kanamycin and gentamycin; metronidazole; clindamycin; and tetracycline (See, e.g., Bradford P. Smith, Large Animal Internal Medicine, 2nd ed. Mosby, St. Louis, 1996 p. 644 and S. Birchard and R. Sherding, Saunders Manual of Small Animal Practice, W.B. Saunders Company, Philadelphia, 1994 p. 739).

Examples of useful antifungal agents include, but are not limited to amphotericin B, ketaconazole, miconazole, 5-fluorocytosine, enilconazole, itraconazole, thiabendazole, and iodides (See, e.g., Bradford P. Smith, Large Animal Internal Medicine, 2nd ed. Mosby, St. Louis, 1996 p. 576 and S. Birchard and R. Sherding, Saunders Manual of Small Animal Practice, W.B. Saunders Company, Philadelphia, 1994 p. 576).

Examples of useful antiviral agents include, but are not limited to, interferon (See, e.g., Bradford P. Smith, Large Animal Internal Medicine, 2nd ed. Mosby, St. Louis, 1996 p. 646).

Examples of useful antiparasitic agents include, but are not limited to, benzimidazoles, such as thiabendazole, fenbendazole, mebendazole, oxfendazole, oxibendazole, albendazole, parbendazole, and febantel; tetrahydropyridines such as morantel tartrate/pyrantel pamoate; levamisole, organophosphates such as haloxon, coumaphos, trichlorfon, and dichlorvos; piperazine salts; ivermectin; and phenothiazine (See, e.g., Bradford P. Smith, Large Animal Internal Medicine, 2nd ed. Mosby, St. Louis, 1996 p. 1688).

Examples of useful antiinflammatory agents include, but are not limited to, corticosteroids such as dexamethasone; antihistamines; and non-steroidal antiinflammatory drugs such as aspirin, flunixin meglumine, phenylbutazone, and ibuprofin (See, e.g., Bradford P. Smith, Large Animal Internal Medicine, 2nd ed. Mosby, St. Louis, 1996 p. 645).

Effective amounts of the other therapeutic agents are known to those skilled in the art. It is well within the skilled artisan's purview to determine the other therapeutic agent's optimal effective-amount range. In one embodiment of the invention, where another therapeutic agent is administered to an animal, the effective amount of a compound of formula 1-6 is less than its effective amount would be were the other therapeutic agent not administered. In this case, without being bound by theory, it is believed that the compound of formula 1-6 and the other therapeutic agent act synergistically to treat or prevent a bacterial infection.

The compound of formula 1-6 and the other therapeutic agent can act additively or, in one embodiment, synergistically. In one embodiment, a compound of formula 1-6 is administered concurrently with another therapeutic agent; for example, a composition comprising an effective amount of a compound of formula 1-6 and an effective amount of another therapeutic agent can be administered. Alternatively, a composition comprising an effective amount of a compound of formula 1-6 and a different composition comprising an effective amount of another therapeutic agent can be concurrently administered. In another embodiment, an effective amount of a compound of formula 1-6 is administered prior or subsequent to administration of an effective amount of another therapeutic agent. In this embodiment, a compound of formula 1-6 is administered while the other therapeutic agent exerts its therapeutic effect, or the other therapeutic agent is administered while the compound of formula 1-6 exerts its therapeutic effect for treating or preventing a bacterial infection.

### Pharmaceutical Compositions Comprising the Compounds of Formula 1-6

Compositions comprising a compound of formula 1-6 can take the form of solutions, suspensions, emulsions, tablets, pills, pellets, capsules, capsules containing liquids, powders, sustained-release formulations, suppositories, aerosols, sprays, suspensions, or any other form suitable for use. In one embodiment, the composition is in the form of a tablet or capsule (See e.g., U.S. Pat. No. 5,698,155).

In one embodiment, the compound of formula 1-6 is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Tablet and pill form are the preferred form for oral delivery. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions are preferred for bacterial infections of the gastrointestinal tract that can cause diarrhea.

In one embodiment, the compound of formula 1-6 is formulated for subcutaneous injection, intramuscular injection, or intravenous administration. Typically, compositions for subcutaneous injection, intramuscular injection, or intravenous administration comprise sterile isotonic aqueous buffer. Where necessary, the compositions can also include a solubilizing agent. Non-aqueous compositions can also be used. Compositions for intravenous administration can optionally include a local anesthetic such as lidocaine to lessen pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of formula 1-6 is to be administered by infusion, they can be dispensed, for example, with an infusion bottle containing sterile pharmaceutical grade water or saline. Where the compound of formula 1-6 is administered by injection, an ampoule of sterile water for injection or saline can be provided so that the ingredients can be mixed prior to administration.

Compositions for oral, subcutaneous injection, intramuscular injection, or intravenous administration typically contain the compound of formula 1-6 in an amount ranging from about 1 percent to 80 percent by weight of the pharmaceutical compositions. In one embodiment, the compositions contain the compound of formula 1-6 in an amount ranging from about 5 percent to 75 percent by weight of the pharmaceutical compositions. In one embodiment, the compositions contain the compound of formula 1-6 in an amount ranging from about 10 percent to 70 percent by weight of the pharmaceutical compositions. In one embodiment, the compositions contain the compound of formula 1-6 in an amount ranging from about 10 percent to 55 percent by weight of the pharmaceutical compositions. In one embodiment, the compositions contain the in an amount ranging from about 15 percent to 65 percent by weight of the pharmaceutical compositions. In one embodiment, the compositions contain therein an amount ranging from about 20 percent to 55 percent by weight of the pharmaceutical compositions.

In another embodiment, this formulated for topical administration. Compositions for topical administration can be in the form of a salve, gel, lotion, cream, or ointment. Compositions for topical administration can be either hydrophilic or hydrophobic and can be aqueous or non-aqueous. Compositions for topical administration can be in the form of an emulsion.

For topical administration, the compositions typically contain a compound of formula 1-6 in an amount ranging from about 0.05 to 10 weight percent of the topical formulation, preferably about 0.05 to 5 weight percent of the topical formulation, more preferably about 0.07 to 4 weight percent of the topical formulation, and most preferably about 0.1 to 3 weight percent of the topical formulation.

The present compositions can optionally comprise a suitable amount of a pharmaceutically acceptable excipient so as to provide the form for proper administration to the animal. Such pharmaceutical excipients can be liquids, such as water, organic solvents, and oils, including those of petroleum, animal, vegetable, or synthetic origin, such as peanut oil, soybean oil, mineral oil, sesame oil, and the like. Pharmaceutically acceptable excipients include, but are not limited to, binding agents, filling agents, lubricating agents, suspending agents, sweeteners, flavoring agents, preservatives, buffers, wetting agents, disintegrants, effervescent agents, coloring agents, pH buffering agents, and other excipients depending upon the route of administration and the dosage form desired. Such excipients are known in the art. Examples of suitable pharmaceutical excipients are described in Remington's Pharmaceutical Sciences 1447-1676 (Alfonso R. Gennaro ed., 19th ed. 1995), the contents of which are incorporated herein by reference.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as fructose, sucrose, xylitol, sodium saccharin, cyclamate, aspartame, and acsulfame. Examples of flavoring agents are Magnasweet® (trademark of MAFCO); oil of wintergreen; bubble gum flavor; peppermint flavor; spearmint flavor; fruit flavors such as cherry, grape, and orange; and the like. Sweetners and flavoring agents are particularly useful in orally administered dosage forms to provide a pharmaceutically palatable preparation.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

In one embodiment, the pharmaceutically acceptable excipients are sterile when administered to an animal. Water, and in one embodiment physiological saline, is a particularly useful excipient when the compound of formula 1-6 is administered intravenously. Saline solutions and aqueous dextrose and glycerol solutions can also be employed as liquid excipients, particularly for injectable solutions. In one embodiment, the liquid excipient is a non-aqueous solvent such as N-methyl-2-pyrollidone; a mixture of N-methyl-2-pyrollidone, polyethylene glycol, and propylene glycol; or the solvents described in U.S. Pat. No. 5,082,863 to Apelian, the contents of which are expressly incorporated herein by reference.

The compositions of the invention are prepared by a method comprising admixing a compound of formula 1-6 and a pharmaceutically acceptable carrier or excipient. Admixing can be accomplished using methods well known for admixing a compound and a pharmaceutically acceptable carrier or excipient.

### Kits

The invention encompasses kits that can simplify the administration of a compound of formula 1-6 to an animal. A typical kit of the invention comprises a unit dosage form of a compound of formula 1-6. In one embodiment, the unit dosage form is a container, which can be sterile, containing an effective amount of a compound of formula 1-6 and a pharmaceutically acceptable carrier or excipient. The kit can further comprise a label or printed instructions instructing the use of the compound of formula 1-6 to treat or prevent a bacterial infection. The kit can also further comprise a unit dosage form of another therapeutic agent, for example, a second container containing an effective amount of the other therapeutic agent and a pharmaceutically acceptable carrier or excipient. In another embodiment, the kit comprises a container containing an effective amount of a compound of formula 1-6, an effective amount of another therapeutic agent, and a pharmaceutically acceptable carrier or excipient. Examples of other therapeutic agents include, but are not limited to, those listed above.

Kits of the invention can further comprise a device that is useful for administering the unit dosage forms. Examples of such a devices include, but are not limited to, a syringe, a drip bag, a dropper, and a patch.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1

### Synthesis of the Compound of Formula 1

The general synthesis of the compound of formula 1 is described in Scheme 1.

Synthesis of Desmycosin: Tylosin tartarate (5.2 g, Sigma-Aldrich, St. Louis Miss.) was dissolved in 50 mL dichloromethane. Dowex (—OH) anion exchange resin was added to neutralize the tartarate. After filtration and rotary evaporation, 4.0 g of tylosin free base was obtained.

Tylosin base (1 g) was dissolved in 225 mL water containing 85 μL 85% phosphoric acid. Sulfuric acid was added dropwise to adjust the pH to approximately 1.6. The solution was heated to 35° C. for 1 hr. The formation of desmycosin was verified by mass spectrometry (M+H=772.8). The reaction mixture was then cooled to room temperature and ethyl acetate (100 mL) was added. While mixing vigorously, concentrated sodium carbonate was slowly added to achieve a basic pH. The organic layer was separated and dried over sodium sulfate. The solvent was removed under reduced pressure to yield approximately 800 mg of solid desmycosin base.

Synthesis of N-methylheptyl amino desmycosin (formula 1): Desmycosin (771 mg, 1 mmol) was dissolve in 25 mL anhydrous tetrahydrofuran (THF) with stirring. N-methylheptyl amine was added (129 mg, 1 mmol) and the mixture warmed to 50° C. for 1 hour. After cooling to room temperature sodium triacetoxyborohydride (1.1 g, 5 eq) was added and the mixture stirred for 2 hours. THF was removed under vacuum and the resulting solid dissolved in 50 mL water. The pH was then adjusted to 4 with acetic acid and 10 g of activated charcoal was added. After 30 minutes, the charcoal was filtered off and 50 mL ethyl acetated was added. The pH was adjusted to 10-11 using sodium carbonate. The organic layer was separated and dried over sodium sulfate. Evaporation of the ethyl acetate yielded approximately 600 mg of product. Recrystallization was achieved using 5-10% ethyl acetate/hexane.

### Example 2

### Synthesis of the Compound of Formula 2

The general synthesis of the compound of formula 2 is described in Scheme 2.

### 2′-Acetyl-Clarithromycin (3)

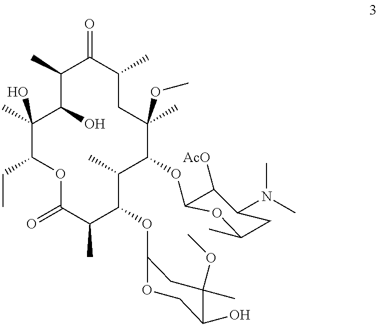

To a solution of clarithromycin (5.0 g, 6.6 mmol) in 80 mL dichloromethane was dropped 4 mL acetyl anhydride in five minute. The mixture was stirred at room temperature for two hours, and then charged on rotary evaporator to remove the solvent. The crude product was re-crystallized in ethyl acetate and hexane (40:60), and the crystal was washed by hexane, and dried under vacuum. The product was obtained as white powder.

ESI-MS (m/z): calculated for C₄₀H₇₁NO₁₄ 789.99. found 791.00 (M+1).

### 4″-DMAP-oxyl-(2′-acetyl) Clarithromycin (4)

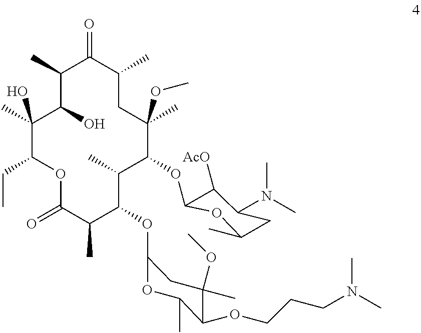

2′-Acetyl clarithromycin (3) (300 mg, 0.38 mmol) was mixed with 1.6 equivalent of N,N-dimethylpropyl bromide hydrobromide (150 mg, 0.60 mmol), KOH pellet (56 mg, 1.0 mmol) in 3 mL dry acetone or acetonitrile. The mixture was stirred at room temperature for 15 hours, and filtered. The filtrate was concentrated via rotary evaporator, and the residue was re-dissolved with 50 mL ethyl acetate followed by washing with water, brine, and dried over anhydrous sodium sulfate. After removing the solvent the residue was subjected to silica gel flash column chromatography, and eluted with gradient elution ethyl acetate/ethanol (100:0 to 0:100, v/v). A combined fraction was concentrated via rotary evaporator, and the residue was re-crystallized in ethyl acetate. A colorless crystal product was obtained, 45%.

ESI-MS (m/z): calculated for C₄₅H₈₂N₂O₁₄ 875.14. found 876.41 {M-(4-dimethylamino-tetrahydro-6-methyl-2H-pyran-3-yl acetate)+1}, 438.80 (M+2).

### 4″-DMAP-Clarithromycin ether (formula 2)

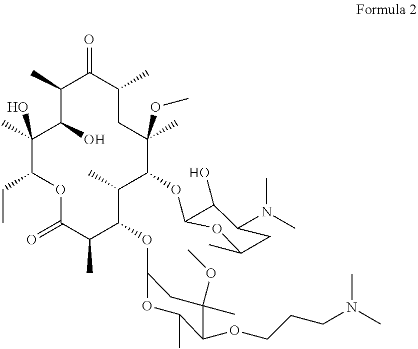

4″-DMAP-oxyl-(2′-acetyl) clarithromycine (4) (250 mg, 0.28 mmol) was dissolved in 20 mL dry methanol, and refluxed under nitrogen for eight hours. After filtered the solvent was removed via rotary evaporator. The residue was re-crystallized in ethyl acetate and hexane (85:15, v/v). A product as white crystal powder was obtained.

ESI-MS (m/z): calculated for C₄₅H₈₂N₂O₁₄ 833.10. found 834.31 (M+1), 672.22 {M-(4-dimethylamino-tetrahydro-6-methyl-2H-pyran-3-ol)+1}, 418.06 (M+2).

### Example 3

### Synthesis of the Compound of Formula 3

The general synthesis of the compound of formula 3 is described in Scheme 3.

### 9-tetrahydrofurfuryl-(4′-DMAP-oxyl)-Clarithromycin oxime (formula 3)

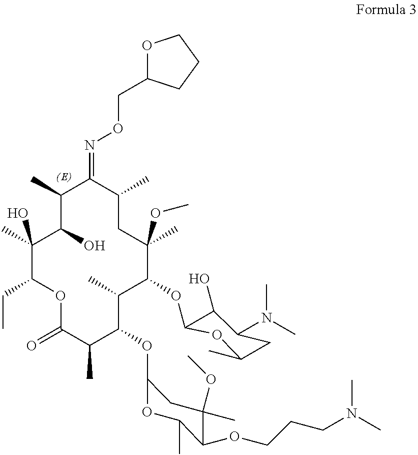

9-(4″-DMAP-oxyl-Clarithromycin) oxime (6) (250 mg, 0.28 mmol) was dissolved in 20 mL dry THF followed by addition of 1.6 equivalents of tetrahydrofurfuryl bromide (93 mg, 0.56 mmol) in 15 mL tetrahydrofuran and potassium hydroxide pellet (25 mg) and anhydrous potassium carbonate (70 mg). The mixture was stirred under nitrogen at room temperature overnight and then filtered and concentrated via rotary evaporation. 30 mL ethyl acetate was added and the aqueous layer retained. The organic layer was washed by 20 mL water once again. To the combined water layer was added concentrated ammonia hydroxyl to adjust pH>10 followed by extracting with dichloride methane (3×20 mL). The combined DCM was washed with brine, dried over anhydrous Na₂SO₄. After filtration the solvent was removed via rotary evaporator. A product as pale white powder was obtained.

ESI-MS (m/z): calculated for C₄₈H₈₉N₃O₁₄ 932.22. found 933.55 (M+1), 774.36 {M-(4-dimethylamino-tetrahydro-6-methyl-2H-pyran-3-ol)+1}, 467.67 (M+2).

### Example 4

### Synthesis of the Compounds of Formula 4 and 5

The general synthesis of the compounds of formula 4 and 5 are described in Scheme 4.

Compound of Formula 5

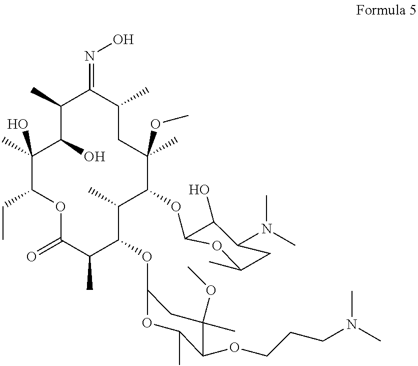

4″-DMAP-oxyl-(2′-acetyl)clarithromycine (4) (500 mg, 0.57 mmol) was dissolved in 150 mL dry methanol. Triethyl amine 3 mL was added, and followed by adding hydroxyl amine hydrochloride. The reaction mixture was refluxed under nitrogen for eight hours. After filtered the solvent was removed via rotary evaporator. The residue was re-crystallized in ethyl acetate and hexane (85:15, v/v). A product as white crystal powder was obtained.

ESI-MS (m/z): calculated for C₄₃H₈₁N₃O₁₃ 848.11. found 849.13 (M+1), 691.02 {M-(4-dimethylamino-tetrahydro-6-methyl-2H-pyran-3-ol)+1}, 425.45 (M+2).

Compound of Formula 4

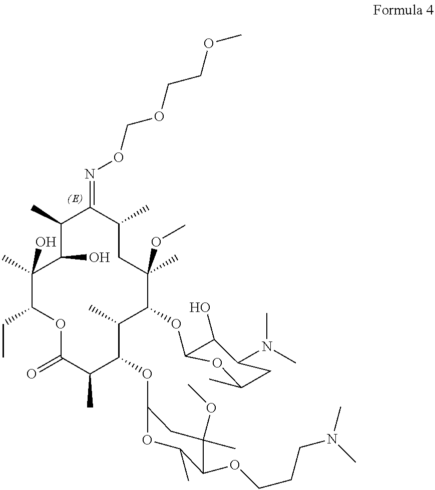

9-(4″-DMAP-oxyl-Clarithromycin) oxime (formula 5) (250 mg, 0.28 mmol) was dissolved in 20 mL dry THF followed by addition of 5.0 equivalents of methoxyethoxymethyl chloride (174 mg, 1.4 mmol) in 15 mL tetrahydrofuran and potassium hydroxide pellet (150 mg) and anhydrous potassium carbonate (420 mg). The mixture was stirred under nitrogen at room temperature overnight and then filtered and concentrated via rotary evaporation. 30 mL ethyl acetate was added and the aqueous layer retained. The organic layer was washed by 20 mL water once again. To the combined water layer was added concentrated ammonia hydroxyl to adjust pH>10 followed by extracting with dichloride methane (3×20 mL). The combined DCM was washed with brine, dried over anhydrous Na₂SO₄. After filtration the solvent was removed via rotary evaporator. A product as pale white powder was obtained.

ESI-MS (m/z): calculated for C₄₇H₈₉N₃O₁₅ 936.22. found 937.55 (M+1), 778.36 {M-(4-dimethylamino-tetrahydro-6-methyl-2H-pyran-3-ol)+1}, 469.67 (M+2).

### Example 5

### Synthesis of the Compound of Formula 6

The general synthesis of the compound of formula 6 is described in Scheme 5.

### Preparation of 3-bromo-N,N-dimethylpropanamine

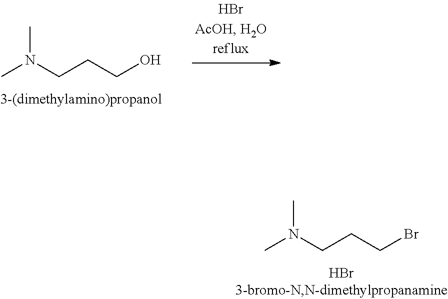

To 7.5 g (72.8 mmol) of 3-dimethylamino-1-propanol was added in 50 mL of acetic acid in ice bath with stirring, followed by adding a solution of HBr in H₂O, 45 g (182 mmol 33%) dropwise. After addition the ice bath was removed, the reaction mixture was heated to reflux for 40 hours. The reaction mixture was charged on vacuum evaporator to remove the most of solvents. To the brownish residue of the crude product was added 200 mL ethanol, and the ethanol was removed via evaporator. The obtained yellow solid salt was re-crystallized in acetone, a needle like yellowish crystal of 3-bromo-N,N-dimethylpropan-amine hydrobromic salt was obtained.

### 9-clarithromycin oxime (1)

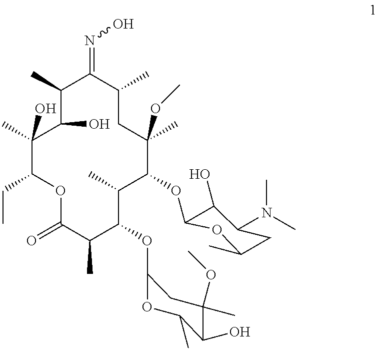

To a solution of clarithromycin (1.0 g, 1.3 mmol) in 15 mL methanol and 10 mL triethylamine was added ten equivalent of hydroxylamine hydrochloride (0.9 g, 13 mmol). The mixture was refluxed with stirring under atmosphere of nitrogen for forty hours. The mixture was filtered after cooled to room temperature, and then followed by adding 5 mL water, and dropping concentrate ammonia hydroxyl to adjust pH to 8. The crystal was filtered, washed with MeOH/H₂O (3:7, v/v) and dried in vacuum. Over 90% E configuration isomer was obtained. Additional product was recovered from mother liquid after concentrated with rotary evaporator. One of major byproduct was a sugar eliminated oxime. The total yield was about 80%.

ESI-MS (m/z): calculated for C₃₈H₇₀N₂O₁₃ 762.97. found 763.89 (M+1).

### 9-DMAP-clarithromycin oxime (formula 6)

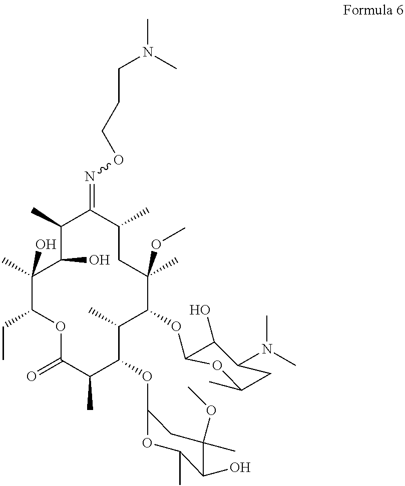

To a solution of clarithromycin oxime (1) (500 mg, 0.65 mmol) and 1.6 equivalent of N,N-dimethylpropyl bromide hydrobromide (670 mg, 1.0 mmol) in 15 mL tetrahydrofuran (THF) was added potassium hydroxide pellet (55 mg) and anhydrous potassium carbonate (138 mg). The mixture of suspension was stirred at room temperature for eight hours, and then filtered. The filtrate was concentrated via rotary evaporator, the residue was re-dissolved by adding 100 mL ethyl acetate, and washed with water (3×100 mL), brine, and the organic layer was dried over anhydrous sodium sulfate. The solvent was removed via rotary evaporator. The colorless residue was re-dissolved in acetone and water (7:3, v/v) to re-crystallize. The crystal was washed by acetone/water (8:2, v/v), and dried in vacuum. Over 97% pure product was obtained in about 90% yield.

ESI-MS (m/z): calculated for C₄₃H₈₁N₃O₁₃ 848.11. found 849.90 (M+1), 691.80 (M-4-dimethylamino-tetrahydro-6-methyl-2H-pyran-3-ol+1), 425.96 (M+2).

A number of references have been cited, the entire disclosures of which are incorporated herein by reference.

What is claimed is:

1. A compound of Formula 1:

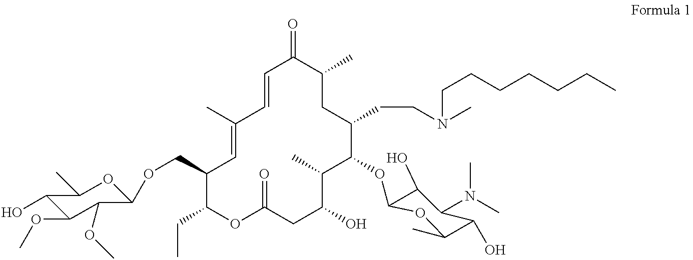

2. A compound of Formula 2:

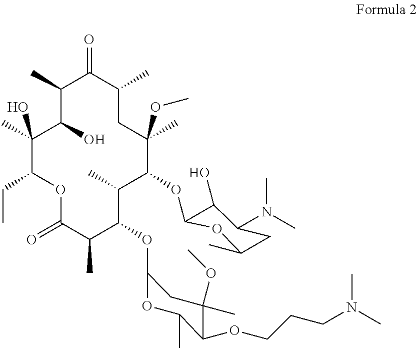

3. A compound of Formula 3:

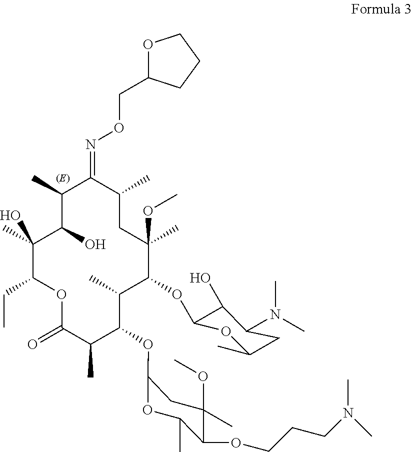

4. A compound of Formula 4:

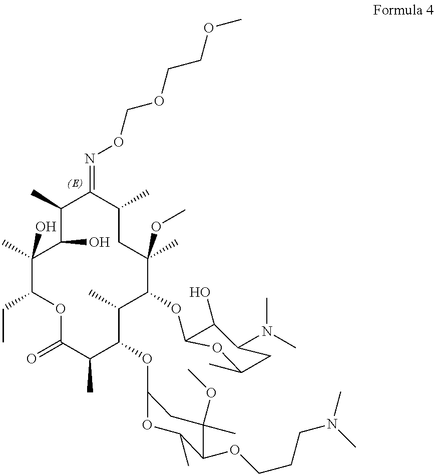

5. A compound of Formula 5:

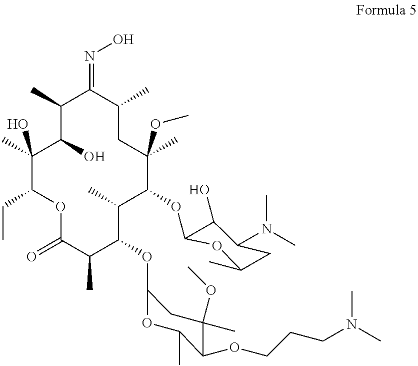

6. A compound of Formula 6:

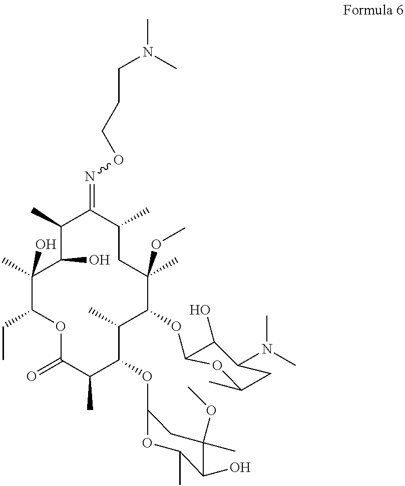

7. A compound selected from the group consisting of:

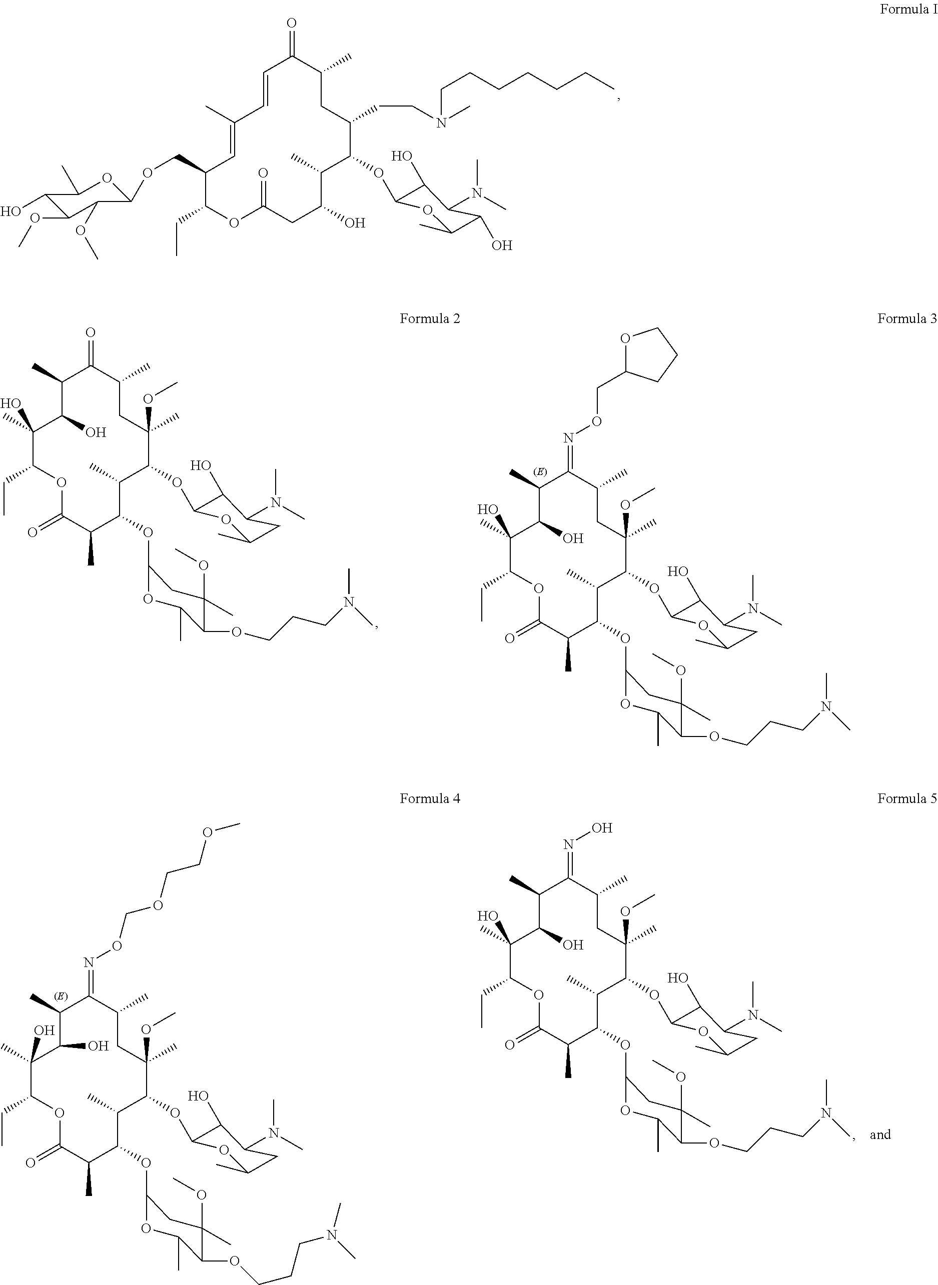

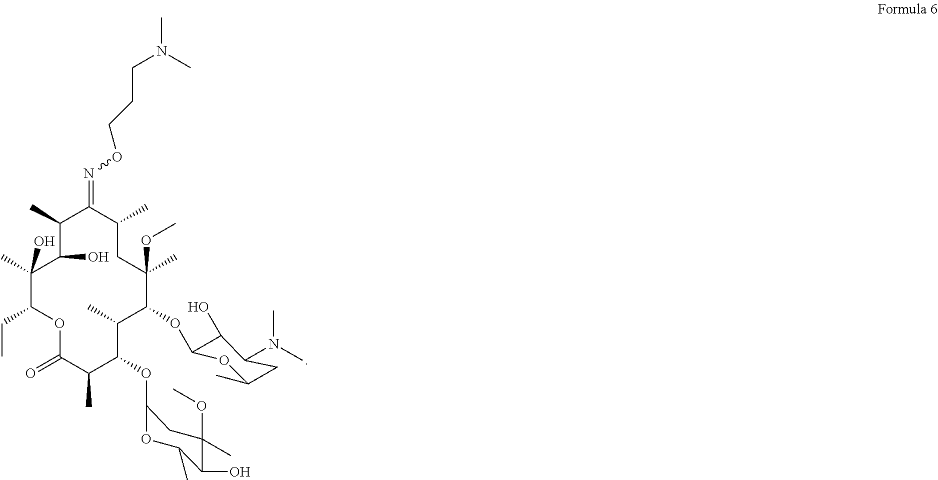

8. A method of treating or preventing an infection comprising administering to an animal in need thereof an amount of a compound of claim 7.

9. The method of claim 8, wherein the administering is by injection.

10. The method of claim 8, wherein the administering is by oral administration.

11. The method of claim 8, wherein the administering is topical administration.

12. The method of claim 8, wherein the infection is a gram positive bacterial infection.

13. The method of claim 8, wherein the infection is a gram negative bacterial infection.

14. The method of claim 8, wherein the amount is about 5 mg/kg/day to about 30 mg/kg/day.

15. The method of claim 8, wherein a single dose of the compound is administered.

19. The method of claim 8, wherein the animal is a mammal.

20. The method of claim 8, wherein the animal a cat, dog, horse, pig, cow, or sheep.

21. The method of claim 8, wherein the animal a human.

22. A pharmaceutical composition comprising a compound of claim 8 and a pharmaceutically acceptable excipient.
